(19) United States
(12) Patent Application Publication
(10) Pub. No.: US 2013/0072688 A1
(43) Pub. Date: Mar. 21, 2013

(54) METHOD FOR PREPARING AN INTERMEDIATE OF PITAVASTATIN OR OF THE SALT THEREOF

(71) Applicants: Seo-Jin Lee, Seoul (KR); Hyun-Gyu Kim, Gyeonggi-do (KR)

(72) Inventors: Seo-Jin Lee, Seoul (KR); Hyun-Gyu Kim, Gyeonggi-do (KR)

(73) Assignee: H.L. GENOMICS, Yongin-si, Gyeonggi-do (KR)

(21) Appl. No.: 13/701,723

(22) PCT Filed: Mar. 11, 2011
(86) PCT No.: PCT/KR2011/001704

§ 371 Date: Dec. 3, 2012

(30) Foreign Application Priority Data
Jun. 8, 2010 (KR) 10-2010-0053631

(51) Int. Cl. C07D 405/02; C07D 405/06; C07D 215/14

(52) U.S. Cl. CPC C07D 405/06; C07D 215/14
USPC 546/173

(57) ABSTRACT

The present invention relates to a method for preparing (4R,6S)-(E)-6-[2-(2-cyclopropyl)-4-(4-fluorophenyl)quinolin-3-yl)-vinyl-2,2-dimethyl-1,3-dioxan-4-yl]acetic acid ester derivative, which is an intermediate of pitavastatin or of the salt thereof, into a crystalline solid form. In addition, the present invention relates to a novel solvate of the intermediate, and to a method for preparing pitavastatin or the salt thereof using the intermediate.

### TECHNICAL FIELD

The present invention relates to an improved process for preparing an intermediate of pitavastatin or its salt. More specifically, the present invention relates to an improved process for preparing an intermediate of pitavastatin or its salt, i.e., (4R,6S)-(E)-6-[2-(2-cyclopropyl)-4-(4-fluorophenyl)quinolin-3-ylyvinyl-2,2-dimethyl-1,3-dioxan-4-yl]acetic acid ester derivatives. And also, the present invention relates to a novel solvate of the intermediate; and to a process for preparing pitavastatin or its salt, using the intermediate.

### BACKGROUND ART

Pitavastatin or its salt, which inhibits cholesterol biosynthesis in the human body through competitive inhibition of 3-hydroxy-3-methyl-glutaryl-coenzyme A (HMG-CoA) reductase, is usefully used for treating hypercholesterolemia. Among the salts of pitavastatin, there are known sodium salt, potassium salt, hemicalcium salt, magnesium salt, etc.; and the hemicalcium salt is being clinically used. The chemical structure of pitavastatin or its salt is as follows, wherein M is hydrogen, Na⁺, K⁺, Mg⁺², Ca⁺² , etc.

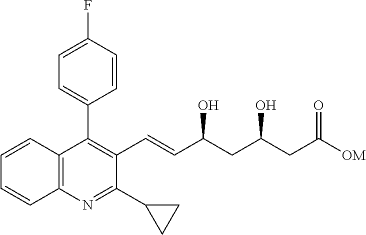

Processes for preparing pitavastatin or its salt have been disclosed in U.S. Pat. Nos. 5,011,930, 5,856,336, 5,872,130, etc. International Publication No. WO 2003/042180 has disclosed an improved process for preparing pitavastatin or its salt. However, because the processes disclosed the above patents include very many reaction steps, give low yield, and use a lithium reagent that is difficult to apply to mass production due to its explosive risk, the processes are difficult to apply to industrial mass production. And also, the prior art processes include using an expensive raw material, trimethylsilane chloride, in high amount, which results in increasing the production cost thereof.

Meanwhile, International Publication No. WO2007/132482 has disclosed a process for preparing pitavastatin or its salt through performing relatively reduced reaction steps under a mild condition. The process disclosed in the patent includes reacting a compound of Formula 2 with tert-butyl-2-((4R,6S)-6-formyl-2,2-dimethyl-1,3-dioxan-4-yl)acetate through Wittig reaction to obtain an intermediate having a dioxane moiety, converting the intermediate to an amine salt form of an intermediate having dihydroxy moieties, and then preparing pitavastatin or its salt, as shown in the following Reaction Scheme 1.

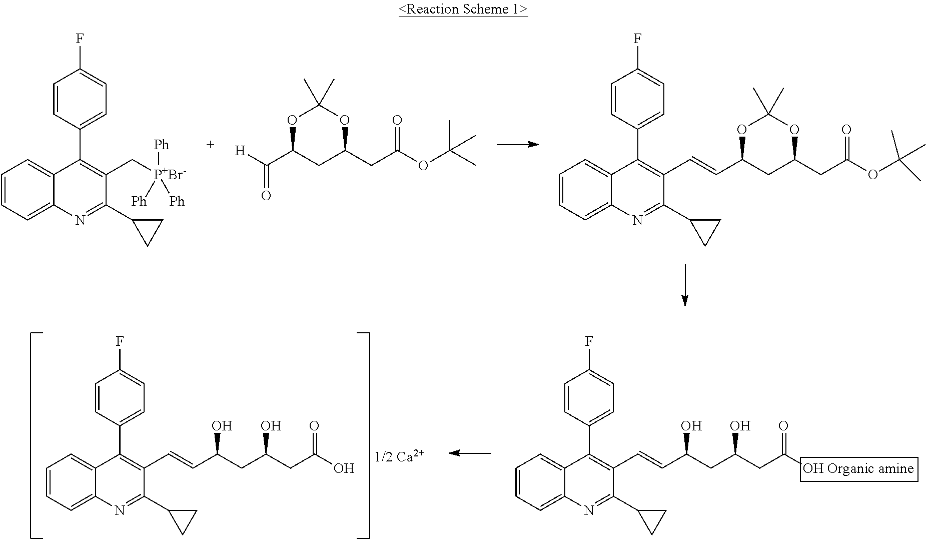

In the above process, the intermediate having a dioxane moiety is isolated by performing isolation steps such as extraction with toluene, concentration, etc. However, such isolation steps give the intermediate in an oil form (i.e., in a concentrated residue form), thereby being difficult to deal therewith. And also, the obtained intermediate having a dioxane moiety has very low purity (about 75% of HPLC purity, see FIG. 1). In addition, the triphenylphosphine oxide originated from the Wittig reaction also resides in an amount of about 2%. In order to address the low purity problems, the above process requires a purification step converting the subsequent product, i.e., the intermediate having dihydroxy moieties to an amine salt form. However, the additional converting step to an amine salt form makes the process long and results in lowering the yield during the conversion. And also, the purity of pitavastatin and its salt obtained from the amine salt form is only HPLC 95% in maximum, which is still unsatisfactory.

Therefore, there remains a need for an improved process for preparing pitavastatin and its salt through short reaction steps under a mild condition, especially a process for preparing pitavastatin and its salt in high purity.

### DETAILED DESCRIPTION OF THE INVENTION

### Technical problem

The present invention provides an improved process for preparing the intermediate having a dioxane moiety in a crystalline solid form through simple methods, without performing complicate post-processing steps such as extraction with an organic solvent, etc. Especially, the present invention provides an improved process for preparing the intermediate in more than 99% of high purity. And also, the present invention provides a process for preparing pitavastatin or its salt in high purity, using the above improved process.

Therefore, the present invention provides an improved process for preparing an intermediate in a crystalline solid form, which is useful for preparing pitavastatin or its salt.

And also, the present invention provides a novel solvate of the intermediate in a crystalline solid form and a process for the preparation thereof.

And also, the present invention provides an improved process for preparing pitavastatin or its salt using the intermediate in a crystalline solid form or its solvate.

### Technical Solution

According to an aspect of the present invention, there is provided a process for preparing a compound of Formula 4 in a crystalline solid form, which comprises: (a) reacting a compound of Formula 2 with a compound of Formula 3 in the presence of a base; and (b) adding C₁˜C₄ alcohol to the reaction mixture of the step (a) to form a precipitate, followed by washing the precipitate with water and then drying to obtain a compound of Formula 4:

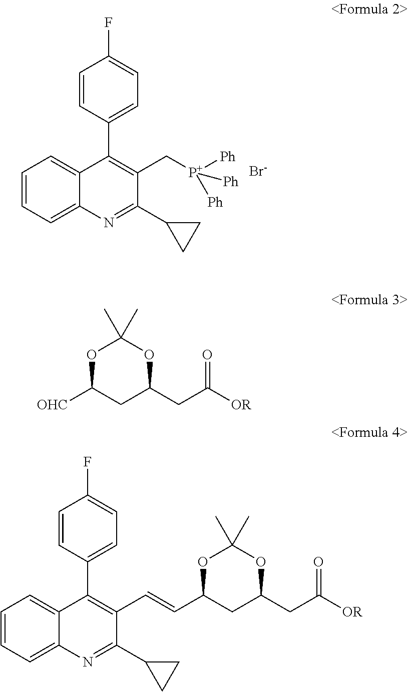

wherein, R is a carboxylic acid-protecting group.

In the process, the base may be an alkali metal salt. The C₁˜C₄ alcohol is one or more selected from the group consisting of methanol, ethanol, and 2-propanol. And also, the forming a precipitate may be performed by adding C₁˜C₄ alcohol to the reaction mixture of the compound of Formula 2 and the compound of Formula 3 at 40 to 45° C.; and then cooling the resulting mixture to 5 to 15° C.

According to another aspect of the present invention, there is provided a process for preparing a dimethyl sulfoxide solvate of the compound of Formula 4 in a crystalline solid form, which comprises: (c) reacting a compound of Formula 2 with a compound of Formula 3 in the presence of a base, using dimethyl sulfoxide as a solvent in a ratio of 3 to 7 L per 1 kg of the compound of Formula 2; and (d) cooling the reaction mixture of the step (c) to 20 to 25° C. to form a precipitate, followed by washing the precipitate with a mixed solvent of dimethyl sulfoxide and hexane and then drying to obtain a compound of Formula 4 in a dimethyl sulfoxide solvate form. In the process, the base may be an alkali metal salt.

According to still another aspect of the present invention, there is provided a dimethyl sulfoxide solvate of the compound of Formula 4;

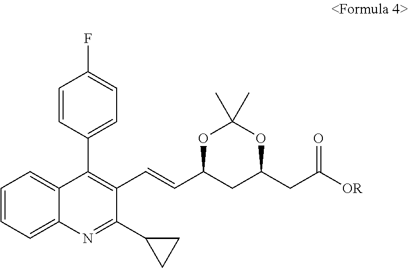

wherein, R is a carboxylic acid-protecting group.

According to still another aspect of the present invention, there is provided a process for preparing pitavastatin or its pharmaceutically acceptable salt, which comprises:

(e) preparing a compound of Formula 4 in a crystalline solid form or its dimethyl sulfoxide solvate according to the above process;

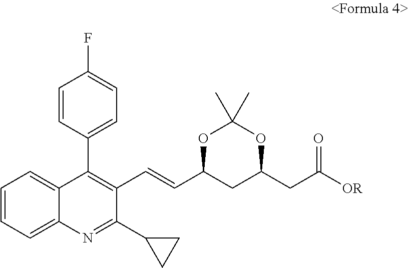

wherein, R is a carboxylic acid-protecting group

(f) adding an acid to the compound of Formula 4 in a crystalline solid form or its dimethyl sulfoxide solvate to give a compound of Formula 5;

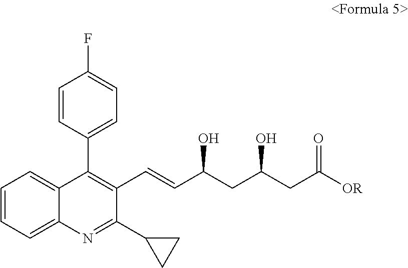

wherein, R is a carboxylic acid-protecting group

(g) adding sodium hydroxide to the compound of Formula 5 to give a free base form of pitavastatin (the compound of Formula 1); and

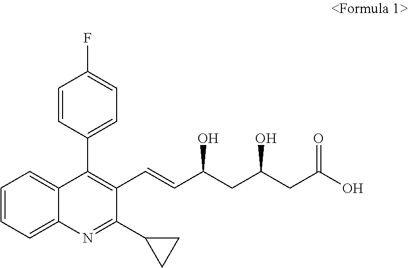 [SMILES: O=C(O)C[C@H](O)C[C@H](O)/C=C/c1c(C2CC2)nc2ccccc2c1-c1ccc(F)cc1]

(h) optionally, converting the free base form of pitavastatin to its pharmaceutically acceptable salt.

### Advantageous Effects

The process according to the present invention can obtain the intermediate (i.e., the compound of Formula 4) or its dimethyl sulfoxide-solvate in a crystalline solid form, thereby avoiding the problems originated from dealing with an oil form of the intermediate. And also, the intermediate in a crystalline solid form or its dimethyl sulfoxide-solvate are obtained in more than 99% of high purity, which makes it possible to prepare a compound of Formula 5 having the dihydroxy moieties in high purity. Therefore, because pitavastatin or its salt can be prepared directly from the compound of Formula 5, without performing the step for converting the compound of Formula 5 to an amine salt form, the process of the present invention can reduce reaction steps, which makes the process suitable for industrial mass production. Especially, because the intermediate can be isolated through crystallizing with a low-priced alcohol or through crystallizing with a small amount of the reaction solvent (i.e., dimethyl sulfoxide) along with temperature-control and washing, without performing isolation steps such as extraction with toluene, concentration, etc. as in the prior art process, the process of the present invention can be simply performed and reduce the production cost.

### BRIEF DESCRIPTION OF THE DRAWINGS

FIG. 1 shows the HPLC analysis result of the product prepared according to Example 3 of International Publication No. WO 2007/132482. In the FIG. 1, the arrow represents the peak of the intermediate having dioxane moiety.

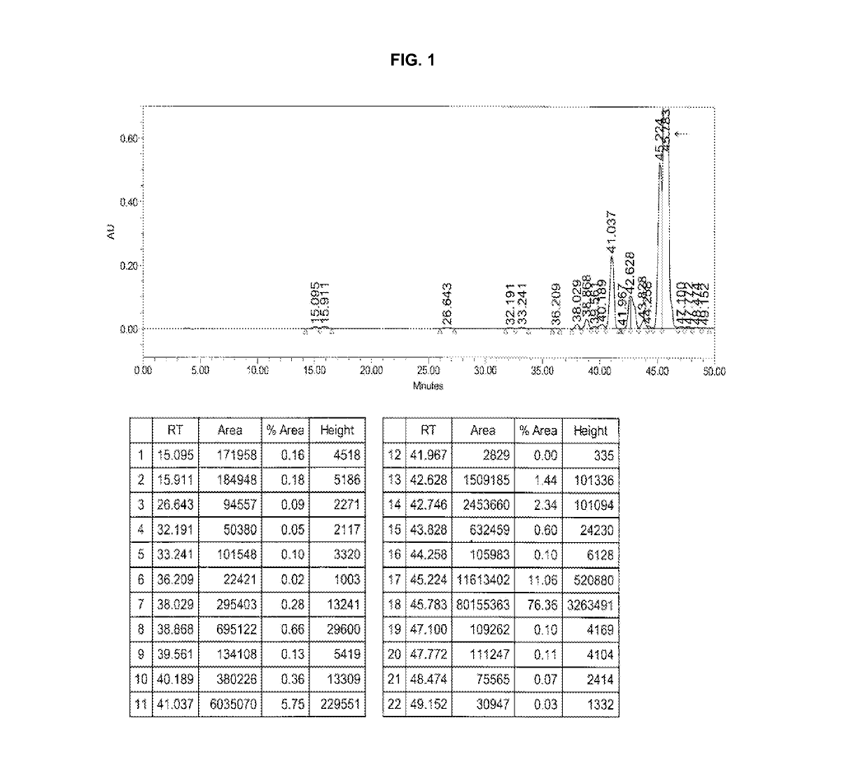

FIG. 2 shows the HPLC analysis result of the product prepared according to Example 1 (the compound of Formula 4, R=tert-butyl). In the FIG. 2, the arrow represents the said product.

### BEST MODE FOR CARRYING OUT THE INVENTION

The present invention provides a process for preparing a compound of Formula 4 in a crystalline solid form, which comprises: (a) reacting a compound of Formula 2 with a compound of Formula 3 in the presence of a base; and (b) adding C₁˜C₄ alcohol to the reaction mixture of the step (a) to form a precipitate, followed by washing the precipitate with water and then drying to obtain a compound of Formula 4:

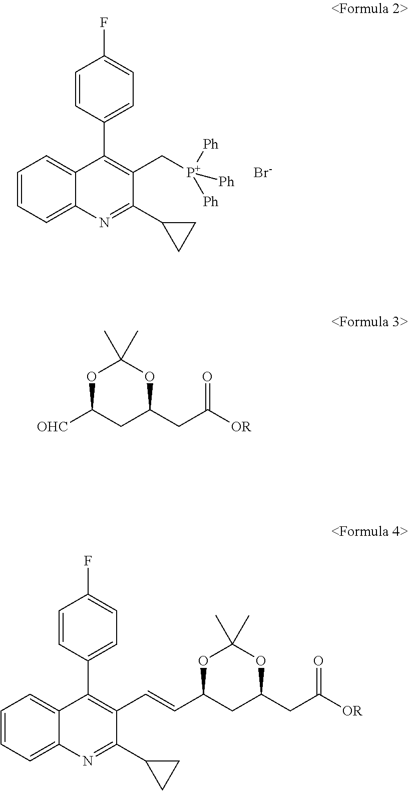

wherein, R is a carboxylic acid-protecting group. The carboxylic acid-protecting group may be a C₁˜C₅ straight or branched alkyl group or a benzyl group; preferably tert-butyl.

The process according to the present invention gives the compound of Formula 4 in a crystalline solid form, thereby avoiding the problems originated from dealing with an oil form of the intermediate (i.e., the compound of Formula 4). And also, the compound of Formula 4 in a crystalline solid form is obtained in more than 99% of high purity, which makes it possible to prepare a compound of Formula 5 having the dihydroxy moieties in high purity. Therefore, because pitavastatin or its salt can be prepared directly from the compound of Formula 5, without performing the step for converting the compound of Formula 5 to an amine salt form as in the prior art process (i.e., International Publication No. WO 2007/132482), the process of the present invention can reduce reaction steps, which makes the process suitable for industrial mass production. Especially, because the intermediate can be isolated through crystallizing with a low-priced alcohol without performing isolation steps such as extraction with toluene, concentration, etc. as in the prior art process, the process of the present invention can be simply performed and reduce the production cost.

The step (a) may be performed according to the prior art process, i.e., International Publication No. WO 2007/132482. That is, the step (a) may be performed by reacting the compound of Formula 2 with the compound of Formula 3 in the presence of a base, wherein the compound of Formula 2 can be obtained from the reaction of 3-(bromomethyl)-2-(1-cyclopropyl)-4-(4′-fluorophenyl)quinoline and triphenylphosphine. The base may be an alkali metal salt, for example sodium hydroxide, potassium hydroxide, sodium carbonate, potassium carbonate, lithium carbonate, cesium carbonate, etc.; preferably, potassium carbonate. The step (a) may be performed at a temperature ranging from 50° C. to 90° C., preferably at about 70° C. The reaction of the compound of Formula 2 and the compound of Formula 3 may be carried out in an organic solvent such as dimethyl sulfoxide, dimethylformamide, tetrahydrofuran, etc.

In the step (b), the C₁˜C₄ alcohol may be one or more selected from the group consisting of methanol, ethanol, and 2-propanol, preferably methanol or ethanol. The addition of the C₁˜C₄ alcohol to the reaction mixture of the step (a) gives a precipitate, which may be isolated by conventional filtration methods. Through forming a precipitate with a C₁˜C₄ alcohol and isolating the precipitate, the by-product, i.e., triphenylphosphine oxide, is removed simply, thereby being able to isolate a crystalline solid form of the compound of Formula 4 in high purity. The forming a precipitate may be performed by adding C₁˜C₄ alcohol to the reaction mixture of the step (a) at 40 to 45°πC.; and then cooling the resulting mixture to 5 to 15° C.

Through the washing process with water in the step (b), the used base is removed. The washing process with water may be performed according to conventional washing methods. For example, the washing process may be performed by using water in a ratio of 5 to 30 L per 1 kg of the precipitate obtained in the previous process. The washing process may be performed one or more. The product obtained from the washing process may be isolated by conventional drying methods, for example drying under reduced pressure.

The present invention also provides a process for preparing a dimethyl sulfoxide solvate of the compound of Formula 4 in a crystalline solid form. That is, the present invention provides a process for preparing a dimethyl sulfoxide solvate of the compound of Formula 4 in a crystalline solid form, which comprises: (c) reacting a compound of Formula 2 with a compound of Formula 3 in the presence of a base, using dimethyl sulfoxide as a solvent in a ratio of 3 to 7 L per 1 kg of the compound of Formula 2; and (d) cooling the reaction mixture of the step (c) to 20 to 25° C. to form a precipitate, followed by washing the precipitate with a mixed solvent of dimethyl sulfoxide and hexane and then drying to obtain a dimethyl sulfoxide solvate form of the compound of Formula 4.

The process for preparing a dimethyl sulfoxide solvate of the compound of Formula 4 according to the present invention gives the compound of Formula 4 in a crystalline solid form, thereby avoiding the problems originated from dealing with an oil form of the intermediate (i.e., the compound of Formula 4). And also, the compound of Formula 4 in a crystalline solid form is obtained in more than 99% of high purity, which makes it possible to prepare a compound of Formula 5 having the dihydroxy moieties in high purity. Therefore, because pitavastatin or its salt can be prepared directly from the compound of Formula without performing the step for converting the compound of Formula 5 to an amine salt form as in the prior art process (i.e., International Publication No. WO 2007/132482), the process of the present invention can reduce reaction steps, which makes the process suitable for industrial mass production. Especially, because the intermediate can be isolated only through crystallizing with a small amount of the reaction solvent (i.e., dimethyl sulfoxide) along with temperature-control and washing, without performing isolation steps such as extraction with toluene, concentration, etc. as in the prior art process, the process of the present invention can be simply performed.

The step (c) may be performed according to prior art process, i.e., International Publication No. WO 2007/132482, except for using dimethyl sulfoxide as a solvent in a small amount, i.e., in a ratio of 3 to 7 L per 1 kg of the compound of Formula 2. The base may be an alkali metal salt, for example sodium hydroxide, potassium hydroxide, sodium carbonate, potassium carbonate, lithium carbonate, cesium carbonate, etc.; preferably, potassium carbonate. The step (c) may be performed at a temperature ranging from 50° C. to 90° C.

The cooling the reaction mixture of the step (c) to 20 to 25° C. gives a precipitate, which may be isolated by conventional filtration methods. Through washing the precipitate with a mixed solvent of dimethyl sulfoxide and hexane, the used base and the by-products, i.e., triphenylphosphine oxide, are removed, thereby being able to isolate a dimethyl sulfoxide solvate of the compound of Formula 4 in a crystalline solid form in high purity. A weight ratio of dimethyl sulfoxide and hexane in the mixed solvent may be 1:0.5 to 1:5, but not limited thereto. The product, i.e., the dimethyl sulfoxide solvate of the compound of Formula 4 in a crystalline solid form, may be isolated by conventional drying methods, for example drying under reduced pressure.

The dimethyl sulfoxide solvate of the compound of Formula 4 in a crystalline solid form is a novel compound, which is useful as an intermediate for preparing pitavastatin or its pharmaceutically acceptable salt. Therefore, the present invention includes, within its scope, the dimethyl sulfoxide solvate of the compound of Formula 4:

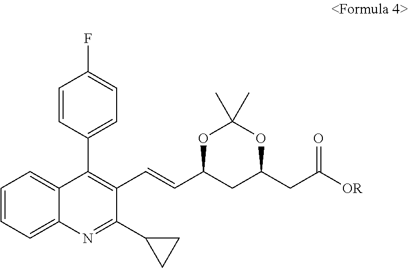

wherein, R is a carboxylic acid-protecting group. The carboxylic acid-protecting group may be a C₁˜C₅ straight or branched alkyl group or a benzyl group; preferably tert-butyl.

The compound of Formula 4 in a crystalline solid form or its dimethyl sulfoxide solvate obtained according to the present invention may be used directly used in the subsequent reaction, without performing separate purification steps or amine-salt converting steps, to prepare pitavastatin or its pharmaceutically acceptable salt in more than 99% of high purity.

Therefore, the present invention also provides a process for preparing pitavastatin or its pharmaceutically acceptable salt, which comprises: (e) preparing a compound of Formula 4 in a crystalline solid form or its dimethyl sulfoxide according to above process; (f) adding an acid to the compound of Formula 4 in a crystalline solid form or its dimethyl sulfoxide solvate to give a compound of Formula 5; (g) adding sodium hydroxide to the compound of Formula 5 to give a free base form of pitavastatin (the compound of Formula 1); and (h) optionally, converting the free base form of pitavastatin to its pharmaceutically acceptable salt:

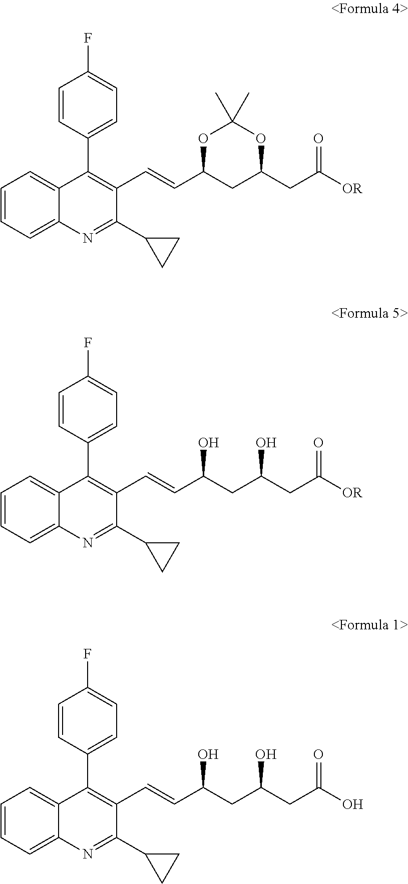

wherein, R is a carboxylic acid-protecting group.

The step (f) may be performed by adding an acid to the compound of Formula 4 in a crystalline solid form or its dimethyl sulfoxide solvate prepared according to the above process to give a compound of Formula 5. The acid treatment results in converting the dioxane moiety to the dihydroxy moieties. The acid may be for example hydrochloric acid, acetic acid, sulfuric acid, etc, preferably hydrochloric acid. The acid may be used in an amount of 1 to 5 equivalents, preferably in an amount of 2 to 3 equivalents, per 1 equivalent of the compound of Formula 4 or its dimethyl sulfoxide solvate, but not limited thereto. The reaction may be performed in a solvent such as acetonitrile, methanol, ethanol, etc. for 2 to 3 hours. The product may be isolated through neutralizing, crystallizing with ethyl acetate and hexane, filtering, drying, etc.

In the step (g), the deprotection by the addition of sodium hydroxide results in converting the compound of Formula 5 to a free base form of pitavastatin. Sodium hydroxide may be used in an amount of 1 to 5 equivalents per 1 equivalent of the compound of Formula 5, but not limited thereto.

The step (h), which is an alternative process, is a process for converting the free base form of pitavastatin to its pharmaceutically acceptable salt. The process may be performed according to prior art processes, e.g., Korean Patent No. 10-0208867. The pharmaceutically acceptable salt of pitavastatin includes conventional salts such as sodium salt, potassium salt, hemicalcium salt, magnesium salt, preferably hemicalcium salt. For example, the converting to hemicalcium salt may be performed by reacting calcium halide such as calcium chloride.

Hereinafter, the present invention will be described more specifically by the following working example. However, the following working example is provided only for illustrations and thus the present invention is not limited to or by it.

### EXAMPLE 1

### Preparation of (4R,6S)-(E)-6-[2-(2-cyclopropyl)-4-(4-fluorophenyl)quinolin-3-yl)-vinyl-2,2-dimethyl-1,3-dioxan-4-yl]acetic acid tert-butyl ester (the compound of Formula 4, R=tert-butyl)

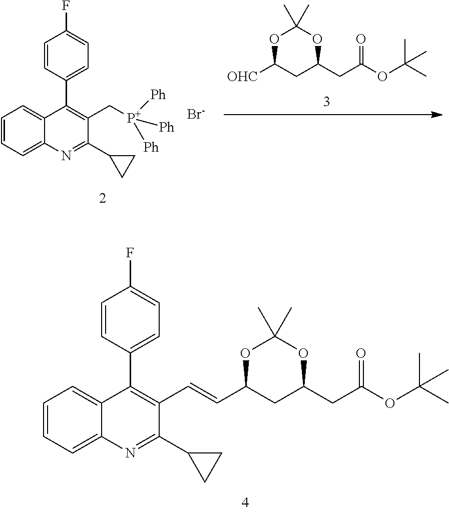 [SMILES: Fc1ccc(-c2c(C[P+](c3ccccc3)(c3ccccc3)c3ccccc3)c(C3CC3)nc3ccccc23)cc1 ; [Br-] ; CC(C)(C)OC(=O)C[C@H]1C[C@@H](C=O)OC(C)(C)O1 ; CC(C)(C)OC(=O)C[C@H]1C[C@@H](/C=C/c2c(C3CC3)nc3ccccc3c2-c2ccc(F)cc2)OC(C)(C)O1]

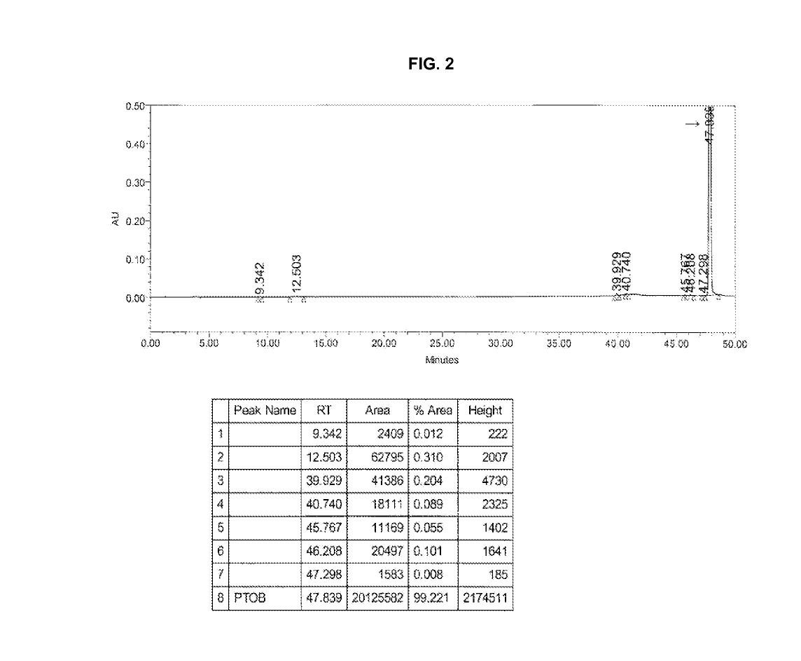

To a solution of tert-butyl-2-((4R,6S)-6-formyl-2,2-dimethyl-1,3-dioxan-4-yl)acetate (the compound of Formula 3, R=tert-butyl) (1.38 kg) in dimethyl sulfoxide (6 L) was added the compound of Formula 2 (3 kg). The reaction mixture was heated to about 35° C. under stirring. K₂CO₃ (1 kg) was added to the resulting clear solution, which was washed with DMSO (3 L). The reaction mixture was heated to about 70° C. and then stirred for 4 hours. After confirming with HPLC that the starting material was exhausted, the reaction was quenched. The reaction mixture was cooled to about 45° C. and then methanol (18 L) was added thereto. The reaction mixture was additionally cooled to about 5° C. and then stirred for additional 2 hours. The resulting precipitate was isolated by filtering under reduced pressure, washed with water (3 L), and then dried under reduced pressure at about 50° C. to give the titled compound (1.82 kg) as a white crystalline form (Yield: 72.3%). The HPLC analysis result of the obtained product is shown in FIG. 2.

Melting point: 113° C.-116° C.

HPLC % Area: 99.396 %

¹H-NMR (CDCl₃): 0.9˜1.0 (m, 2H), 1.0˜1.03(m, 1H), 1.2˜1.3(m, 2H), 1.3˜1.38(s, 6H), 1.38˜1.4(m, 1H), 1.45(s, 9H), 2.2˜2.3(dd, 1H, J=0.015, J=0.038), 2.3˜2.4 (m, 2H), 4.1˜4.2(m, 1H), 4.3˜4.4(m, 1H), 5.5˜5.6(dd, 1H, J=0.015, J=0.04), 6.5(d, 1H, J=0.04), 7.0˜7.3(m, 6H), 7.5(t, 1H), 7.9(d, 1H, J=0.021)

### EXAMPLE 2

### Preparation of (4R,6S)-(E)-6-[2-(2-cyclopropyl)-4-(4-fluorophenyl)quinolin-3-yl)-vinyl-2,2-dimethyl-1,3-dioxan-4-yl]acetic acid tert-butyl ester (the compound of Formula 4, R=tert-butyl)

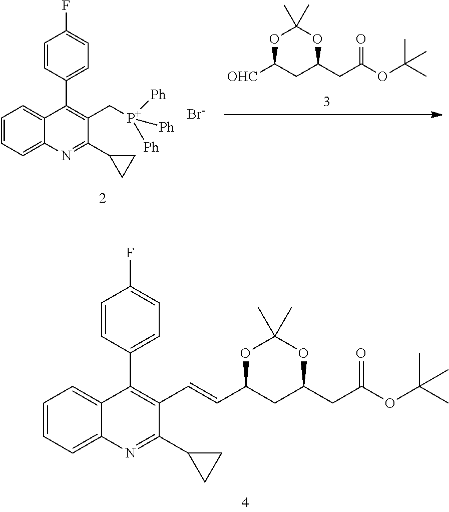 [SMILES: Fc1ccc(-c2c(C[P+](c3ccccc3)(c3ccccc3)c3ccccc3)c(C3CC3)nc3ccccc23)cc1 ; [Br-] ; CC(C)(C)OC(=O)C[C@H]1C[C@@H](C=O)OC(C)(C)O1 ; CC(C)(C)OC(=O)C[C@H]1C[C@@H](/C=C/c2c(C3CC3)nc3ccccc3c2-c2ccc(F)cc2)OC(C)(C)O1]

To a solution of tert-butyl-2-((4R,6S)-6-formyl-2,2-dimethyl-1,3-dioxan-4-yl)acetate (the compound of Formula 3, R=tert-butyl) (1.38 kg) in dimethylformamide (4 L) was added the compound of Formula 2 (3 kg). The reaction mixture was heated to about 35° C. under stirring. K₂CO₃ (1 kg) was added to the resulting clear solution, which was washed with dimethylformamide (1.5 L). The reaction mixture was heated to about 90° C. and then stirred for 2 hours. After confirming with HPLC that the starting material was exhausted, the reaction was quenched. The reaction mixture was cooled to about 45° C. and then methanol (10 L) was added thereto. The reaction mixture was additionally cooled to about 5° C. and then stirred for additional 2 hours. The resulting precipitate was isolated by filtering under reduced pressure, washed with water (3 L), and then dried under reduced pressure at about 50° C. to give the titled compound (1.58 kg) as a white crystalline form (Yield: 63.0%).

### EXAMPLE 3

### Preparation of (4R,6S)-(E)-6-[2-(2-cyclopropyl)-4-(4-fluorophenyl)quinolin-3-yl)-vinyl-2,2-dimethyl-1,3-dioxan-4-yl]acetic acid tert-butyl ester (the compound of Formula 4, R=tert-butyl)

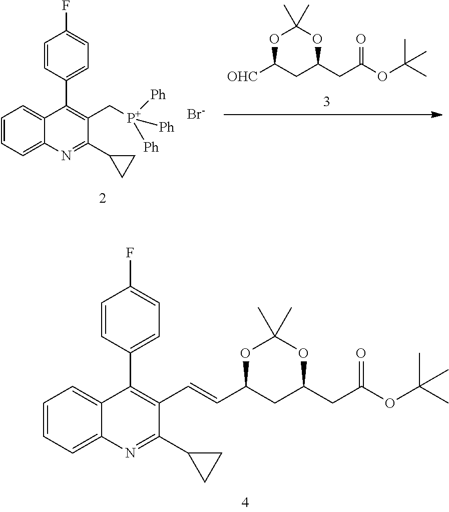 [SMILES: Fc1ccc(-c2c(C[P+](c3ccccc3)(c3ccccc3)c3ccccc3)c(C3CC3)nc3ccccc23)cc1 ; [Br-] ; CC(C)(C)OC(=O)C[C@H]1C[C@@H](C=O)OC(C)(C)O1 ; CC(C)(C)OC(=O)C[C@H]1C[C@@H](/C=C/c2c(C3CC3)nc3ccccc3c2-c2ccc(F)cc2)OC(C)(C)O1]

To a solution of tert-butyl-2-((4R,6S)-6-formyl-2,2-dimethyl-1,3-dioxan-4-yl)acetate (the compound of Formula 3, R=tert-butyl) (1.38 kg) in dimethyl sulfoxide (6 L) was added the compound of Formula 2 (3 kg). The reaction mixture was heated to about 35° C. under stirring. K₂CO₃ (1 kg) was added to the resulting clear solution, which was washed with DMSO (3 L). The reaction mixture was heated to about 70° C. and then stirred for 4 hours. After confirming with HPLC that the starting material was exhausted, the reaction was quenched. The reaction mixture was cooled to about 45° C. and then ethanol (18 L) was added thereto. The reaction mixture was additionally cooled to about 5° C. and then stirred for additional 2 hours. The resulting precipitate was isolated by filtering under reduced pressure, washed with water (3 L), and then dried under reduced pressure at about 50° C. to give the titled compound (1.76 kg) as a white crystalline form (Yield: 70.1%).

### EXAMPLE 4

### Preparation of DMSO-solvate of (4R,6S)-(E)-6-[2-(2-cyclopropyl)-4-(4-fluorophenyl)quinolin-3-yl)-vinyl-2,2-dimethyl-1,3-dioxan-4-yl]acetic acid tert-butyl ester (the compound of Formula 4, R=tert-butyl)

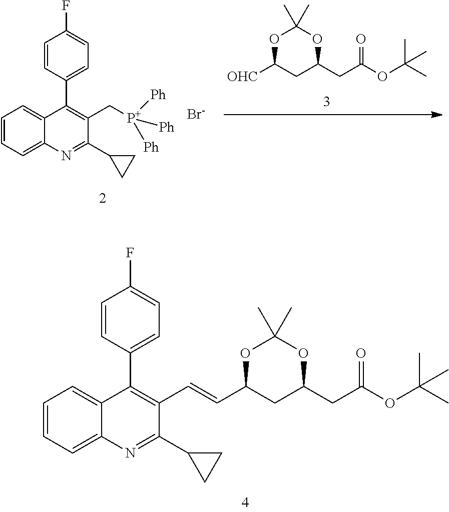 [SMILES: Fc1ccc(-c2c(C[P+](c3ccccc3)(c3ccccc3)c3ccccc3)c(C3CC3)nc3ccccc23)cc1 ; [Br-] ; CC(C)(C)OC(=O)C[C@H]1C[C@@H](C=O)OC(C)(C)O1 ; CC(C)(C)OC(=O)C[C@H]1C[C@@H](/C=C/c2c(C3CC3)nc3ccccc3c2-c2ccc(F)cc2)OC(C)(C)O1]

To a solution of tert-butyl-2-((4R,6S)-6-formyl-2,2-dimethyl-1,3-dioxan-4-yl)acetate (the compound of Formula 3, R=tert-butyl) (1.38 kg) in dimethyl sulfoxide (10 L) was added the compound of Formula 2 (3 kg). The reaction mixture was heated to about 35° C. under stirring. K₂CO₃ (1 kg) was added to the resulting clear solution, which was washed with DMSO (8 L). The reaction mixture was heated to about 70° C. and then stirred for 4 hours. After confirming with HPLC that the starting material was exhausted, the reaction was quenched. The reaction mixture was slowly cooled to about 25° C. and then stirred for 6 hours. The resulting precipitate was isolated by filtering under reduced pressure, washed with a mixed solvent of dimethyl sulfoxide and hexane (2:1, weigh ratio) (5 L), and then dried under reduced pressure at about 50° C. to give the titled compound (DMSO-solvate) (2.15 kg) as a white crystalline form (Yield: 65.7%).

Melting point: 97° C.-103° C.

HPLC % Area: 99.221%

¹H-NMR (CDCl₃): 0.9˜1.0 (m, 2H), 1.0˜1.03(m, 1H), 1.2˜1.3(m, 2H), 1.3˜1.38(s, 6H), 1.38˜1.4(m, 1H), 1.45(s, 9H), 2.2˜2.3(dd, 1H, J=0.015, J=0.038), 2.3˜2.4 (m, 2H), 2.5˜2.7(m, 12H), 4.1˜4.2(m, 1H), 4.3˜4.4(m, 1H), 5.5˜5.6(dd, 1H, J=0.015, J=0.04), 6.5(d, 1H, J=0.04), 7.0˜7.3(m, 6H), 7.5(t, 1H), 7.9(d, 1H, J=0.021)

### EXAMPLE 5

### Preparation of (4R,6S)-(E)-6-[2-(2-cyclopropyl)-4-(4-fluorophenyl)quinolin-3-yl)-vinyl-2,2-dimethyl-1,3-dihydroxy-4-yl]acetic acid tert-butyl ester (the compound of Formula 5, R=tert-butyl)

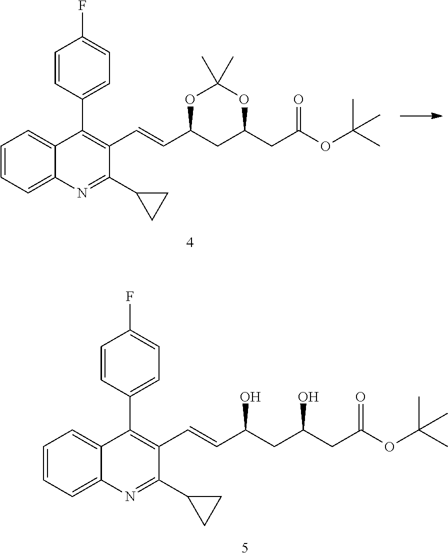 [SMILES: CC(C)(C)OC(=O)C[C@H]1C[C@@H](/C=C/c2c(C3CC3)nc3ccccc3c2-c2ccc(F)cc2)OC(C)(C)O1 ; CC(C)(C)OC(=O)C[C@H](O)C[C@H](O)/C=C/c1c(C2CC2)nc2ccccc2c1-c1ccc(F)cc1]

The compound prepared in Example 1 (1.5 kg) was added to acetonitrile (16 L). A mixed solution of a 35% HCl solution (0.91 kg) and purified water (9.5 kg) was slowly added over 2 hours to the mixture under stirring. The reaction mixture was stirred for additional 1 hour. After confirming with HPLC that the starting material was exhausted, the reaction was quenched. The reaction mixture was neutralized with sodium bicarbonate and then extracted with ethyl acetate. The separated organic layer was washed with a sodium chloride solution (1.5 kg) and then concentrated under reduced pressure. The resulting residue was dissolved in ethyl acetate (1.5 L) and then hexane (9 L) was slowly added thereto. The reaction mixture was cooled to about 10° C. and then stirred for 2 hours. The resulting precipitate was isolated by filtering under reduced pressure and then dried under reduced pressure at about 50° C. to give the titled compound (1.22 kg) as a white crystalline form (Yield: 88.4%).

HPLC % Area: 98.555%

### EXAMPLE 6

### Preparation of (4R,6S)-(E)-6-[2-(2-cyclopropyl)-4-(4-fluorophenyl)quinolin-3-yl)-vinyl-2,2-dimethyl-1,3-dihydroxy-4-yl]acetic acid tert-butyl ester (the compound of Formula 5, R=tert-butyl)

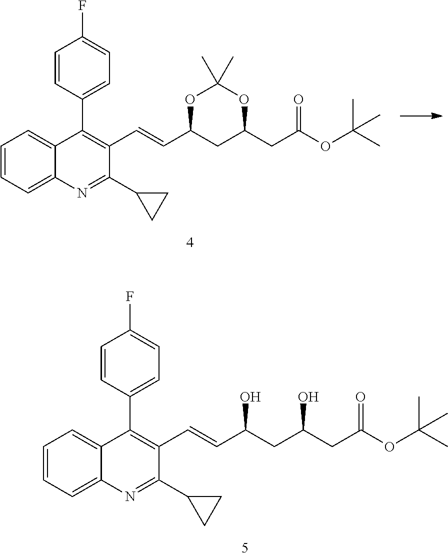 [SMILES: CC(C)(C)OC(=O)C[C@H]1C[C@@H](/C=C/c2c(C3CC3)nc3ccccc3c2-c2ccc(F)cc2)OC(C)(C)O1 ; CC(C)(C)OC(=O)C[C@H](O)C[C@H](O)/C=C/c1c(C2CC2)nc2ccccc2c1-c1ccc(F)cc1]

The compound (DMSO-solvate) prepared in Example 4 (2.15 kg) was added to acetonitrile (16 L). A mixed solution of a 35% HCl solution (1.1 kg) and purified water (12.9 kg) was slowly added over 2 hours to the mixture under stirring. The reaction mixture was stirred for additional 1 hour. After confirming with HPLC that the starting material was exhausted, the reaction was quenched. The reaction mixture was neutralized with sodium bicarbonate and then extracted with ethyl acetate. The separated organic layer was washed with a sodium chloride solution (2.15 kg) and then concentrated under reduced pressure. The resulting residue was dissolved in ethyl acetate (2.15 L) and then hexane (12.9 L) was slowly added thereto. The reaction mixture was cooled to about 10° C. and then stirred for 2 hours. The resulting precipitate was isolated by filtering under reduced pressure and then dried under reduced pressure at about 50° C. to give the titled compound (1.56 kg) as a white crystalline form (Yield: 78.79%).

HPLC % Area : 98.615%

### EXAMPLE 7

### Preparation of pitavastatin hemicalcium Salt

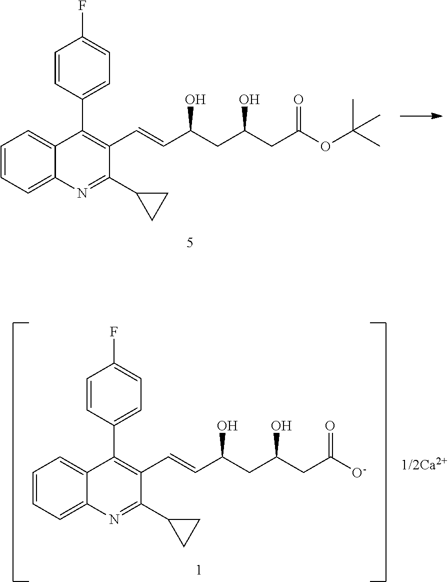

The compound prepared in Example 5 (1.2 kg) was added to purified water (19 kg). A solution of sodium hydroxide (0.08 kg) in purified water (1 kg) was slowly added to the mixture under stirring. The reaction mixture was stirred for 1 hour at room temperature. A solution of calcium chloride (0.28 kg, purity: 95%) in purified water (1 kg) was slowly added at room temperature over 2 hours to the reaction mixture, which was then stirred at the same temperature for additional 1 hour. The resulting precipitate was isolated by filtering under reduced pressure and then dried under reduced pressure at about 40° C. to give pitavastatin hemicalcium salt (1.0 kg) as a white solid form (Yield: 88.5%).

HPLC % Area : 99.826%

### EXAMPLE 8

### Preparation of pitavastatin hemicalcium Salt

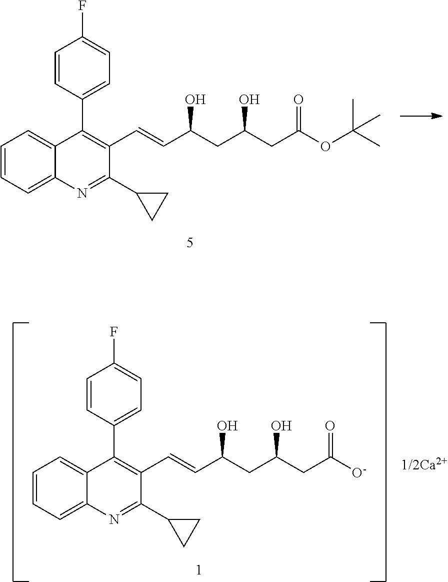

The compound prepared in Example 6 (1.56 kg) was added to purified water (25 kg). A solution of sodium hydroxide (0.26 kg) in purified water (1.5 kg) was slowly added to the mixture under stirring. The reaction mixture was stirred for 1 hour at room temperature. A solution of calcium chloride (0.36 kg, purity: 95%) in purified water (1.5 kg) was slowly added at room temperature over 2 hours to the reaction mixture, which was then stirred at the same temperature for additional 1 hour. The resulting precipitate was isolated by filtering under reduced pressure and then dried under reduced pressure at about 40° C. to give pitavastatin hemicalcium salt (1.2 kg) as a white solid form (Yield: 83.3%).

HPLC % Area : 99.776%

1. A process for preparing a compound of Formula 4 in a crystalline solid form, which comprises:
(a) reacting a compound of Formula 2 with a compound of Formula 3 in the presence of a base; and
(b) adding C₁˜C₄ alcohol to the reaction mixture of step (a) to form a precipitate, followed by washing the precipitate with water and then drying to obtain a compound of Formula 4: 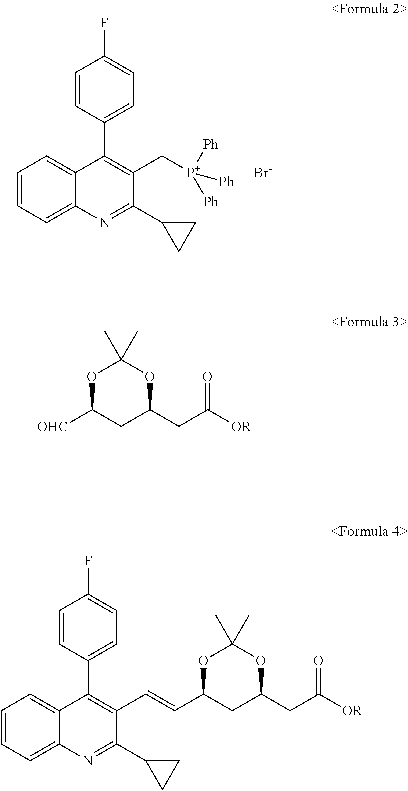
wherein, R is a carboxylic acid-protecting group.

2. A process for preparing a dimethyl sulfoxide solvate of the compound of Formula 4 in a crystalline solid form, which comprises:
(c) reacting a compound of Formula 2 with a compound of Formula 3 in the presence of a base, using dimethyl sulfoxide as a solvent in a ratio of 3 to 7 L per 1 kg of the compound of Formula 2; and
(d) cooling the reaction mixture of step (c) to 20 to 25° C. to form a precipitate, followed by washing the precipitate with a mixed solvent of dimethyl sulfoxide and hexane and then drying to obtain a dimethyl sulfoxide solvate form of the compound of Formula 4: 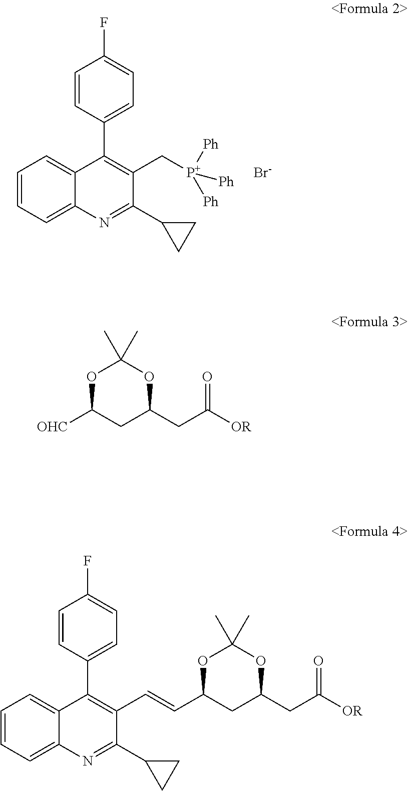
wherein, R is a carboxylic acid-protecting group.

3. The process of claim 1, wherein the base is an alkali metal salt.

4. The process of claim 1, wherein the C₁˜C₄ alcohol is one or more selected from the group consisting of methanol, ethanol, and 2-propanol.

5. The process of claim 1, wherein the forming a precipitate is performed by adding C₁˜C₄ alcohol to the reaction mixture of the compound of Formula 2 and the compound of Formula 3 at 40 to 45° C.; and then cooling the resulting mixture to 5 to 15° C.

6. A dimethyl sulfoxide solvate of the compound of Formula 4; 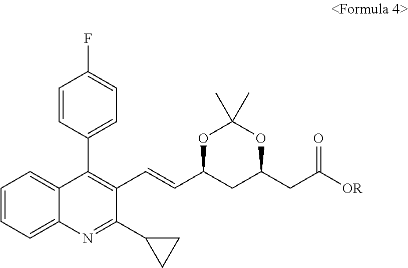
wherein, R is a carboxylic acid-protecting group.

7. A process for preparing pitavastatin or its pharmaceutically acceptable salt, which comprises:
(e) preparing a compound of Formula 4 in a crystalline solid form or its dimethyl sulfoxide solvate according to claim 1; 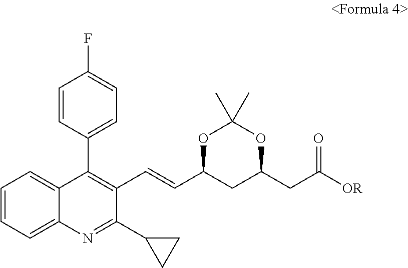
wherein, R is a carboxylic acid-protecting group
(f) adding an acid to the compound of Formula 4 in a crystalline solid form or its dimethyl sulfoxide solvate to give a compound of Formula 5; 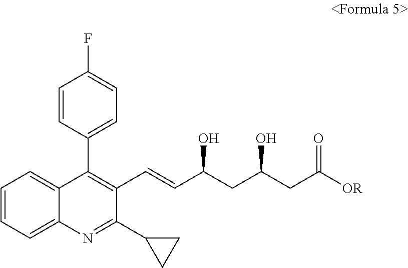
wherein, R is a carboxylic acid-protecting group
(g) adding sodium hydroxide to the compound of Formula 5 to give a free base form of pitavastatin; and
(h) optionally, converting the free base form of pitavastatin to its pharmaceutically acceptable salt.

8. The process of claim 2, wherein the base is an alkali metal salt.

9. A process for preparing pitavastatin or its pharmaceutically acceptable salt, which comprises:
(e) preparing a compound of Formula 4 in a crystalline solid form or its dimethyl sulfoxide solvate according to claim 2; 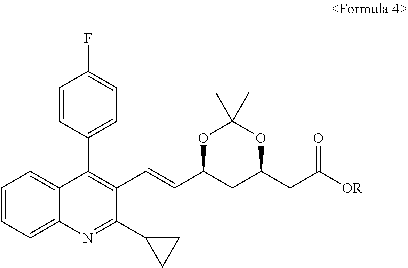
wherein, R is a carboxylic acid-protecting group
(f) adding an acid to the compound of Formula 4 in a crystalline solid form or its dimethyl sulfoxide solvate to give a compound of Formula 5; 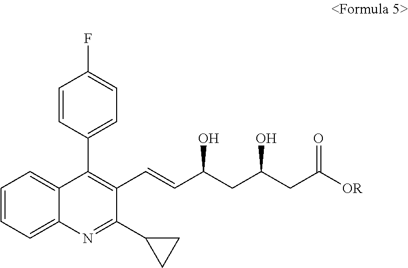
wherein, R is a carboxylic acid-protecting group
(g) adding sodium hydroxide to the compound of Formula 5 to give a free base form of pitavastatin; and
(h) optionally, converting the free base form of pitavastatin to its pharmaceutically acceptable salt.
